(19) United States
(12) Patent Application Publication
(10) Pub. No.: US 2014/0194379 A1
(43) Pub. Date: Jul. 10, 2014

(54) METHODS FOR THE PRODUCTION OF CHITIN NANOFIBERS AND USES THEREOF

(71) Applicant: University of Washington Through Its Center For Commercialization, Seattle, WA (US)

(72) Inventors: Marco Rolandi, Seattle, WA (US); Ronald Berenson, Mercer Island, WA (US); Vittorio Ruvolo, Novi Ligure (AL) (IT); Adnan Kapetanovic, Tukwila, WA (US); Jungho Jin, Seattle, WA (US); Chao Zhong, Bedford, MA (US)

(73) Assignee: University of Washington Through Its Center For Commercialization, Seattle, WA (US)

(21) Appl. No.: 14/123,726

(22) PCT Filed: Jun. 1, 2012
(86) PCT No.: PCT/US12/40565

§ 371 Date: Mar. 17, 2014

Related U.S. Application Data
(60) Provisional application No. 61/492,927, filed on Jun. 3, 2011.
(60) Provisional application No. 61/558,372, filed on Nov. 10, 2011.

(51) Int. Cl. A61L 31/04; A61K 8/73; A61M 37/00

(52) U.S. Cl. CPC A61L 31/042; A61M 37/0015; A61K 8/736
USPC 514/55; 604/173; 536/20; 427/243

(57) ABSTRACT

Methods for the production chitin nanofibers and uses thereof. Furthermore, methods for the production of chitin nanofibers and the fabrication of chitin nanofiber structures and devices.

### TECHNICAL FIELD

This disclosure relates to methods for the production chitin nanofibers and uses thereof. More specifically, this disclosure relates to methods for the production of chitin nanofibers and the fabrication of chitin nanofiber based structures and devices.

### BACKGROUND

Chitin is a polymer of N-acetylglucosamine (poly(β-(1-4)-N-acetyl-D-glucosamine)) that is present in various marine and terrestrial organisms, including cephalopods, crustacea, insects, mollusks, and also in microorganisms, algae, plants, and fungi. Chitin is abundant, biocompatible, biodegradable, nontoxic, and physiologically inert. Chitin also has beneficial wound healing properties such as anti-bacterial activity, prevention of bleeding, decreasing inflammation, and reducing scarring.

The physical and mechanical properties of chitin can be modified according to the desired application. For example, deacetylation of the chitin polymer may be used to produce chitosan, a derivative of chitin. Moreover, chitin may be formed into chitin nanofibers that may be used in composites and biomaterials with medical and pharmaceutical applications in areas such as wound care devices, controlled drug release, tissue engineering, hemostatic agents, anticoagulants, antiviral agents, dialysis membranes, orthopedic materials, etc.

The production of chitin nanofibers is challenging because of chitin's intractability and water insolubility. Furthermore, conventional approaches to the production of chitin nanofibers either rely upon top-down procedures that break down the starting bulk material in harsh conditions, or involve electrospinning of depolymerized chitin solutions. Such approaches tend to degrade the polymer and hamper its natural properties. Additionally, these production methods may use highly basic or highly acidic environments with mechanical forces that result in deacetylated or depolymerized chitin fibers.

### BRIEF DESCRIPTION OF THE DRAWINGS

FIG. 1 is a schematic representing certain embodiments of methods for the production of chitin nanofibers.

FIG. 2 shows chitin nanofiber micrograph images of the morphology and diameter distribution of chitin nanofibers produced according to the methods disclosed herein.

FIG. 3 shows micrograph images of the effects of chitin solution concentration on chitin nanofiber morphology produced according to the methods disclosed herein (scale bars=200 nm).

FIG. 4 shows micrograph images of chitin nanofibers produced from different solvent evaporation rates.

FIG. 5 is a schematic representation of the fabrication of chitin nanofiber structures according the methods disclosed herein.

FIG. 6 shows micrograph images and analysis of chitin nanofiber films fabricated according the methods disclosed herein.

FIG. 7 shows micrograph images and analysis of chitin nanofiber films fabricated according the methods disclosed herein.

FIG. 8 shows micrograph images of chitin nanofiber structures fabricated according the methods disclosed herein.

FIG. 9 is a schematic diagramming one embodiment of a method of chitin nanofiber micromolding as disclosed herein.

FIG. 10 shows micrograph images of micromolded chitin nanofiber structures fabricated according to the methods disclosed herein.

FIG. 11 is a schematic diagramming one embodiment a method of chitin nanofiber printing as disclosed herein.

FIG. 12 shows micrograph images of printed chitin nanofiber structures produced according to the methods disclosed herein.

FIG. 13 is a schematic diagramming one embodiment of the fabrication of replica molds for the production of chitin nanofiber microneedles according to the methods disclosed herein.

FIG. 14 is a schematic diagram showing the production a chitin nanofiber microneedle array according to the methods disclosed herein.

FIG. 15 shows SEM images of chitin nanofiber microneedle arrays produced according to the methods disclosed herein.

FIG. 16 is a drawing representing different embodiments of chitin nanofiber microneedles fabricated according to the methods disclosed herein.

### DETAILED DESCRIPTION

I. Definitions

The terminology used herein is for the purpose of describing particular embodiments only, and is not intended to limit the scope of the present invention, which will be limited only by the appended claims. Unless defined otherwise, all technical and scientific terms used herein have the meanings that would be commonly understood by one of skill in the art in the context of the present specification.

As used herein, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the terms “approximately” and “about,” as used herein when referring to a measurable value such as an amount, dose, time, temperature, and the like, is meant to encompass variations of 20%, 10%, 5%, 1%, 0.5%, or even 0.1% of the specified amount.

Unless otherwise indicated, the term “include” has the same meaning as “include, but are not limited to,” the term “includes” has the same meaning as “includes, but is not limited to,” and the term “including” has the same meaning as “including, but not limited to.” Similarly, the term “such as” has the same meaning as the term “such as, but not limited to.”

As used herein, the term “wound care device” means a device used for closing a wound, covering a wound, protecting a wound, a wound dressing, a bandage, etc.

As used herein, the term “wound” means an injury to tissue or skin caused by a cut, surgical procedure, tear, laceration, piercing, trauma, or other impact. For example, a wound may be an incision performed during a surgical procedure or operation. In another example, a wound may include ulcers, such as diabetic ulcers, ulcers from vascular insufficiency, pressure sores, and burns.

As used herein, the term “tissue” means any human or other animal tissue including skin, muscle, tendon, bone, heart, lung, kidney, brain, bowel, colon, rectum, stomach, esophagus, etc.

As used herein, the term weight percent (wt %) is a way of expressing the concentration of a solution and is calculated by dividing the weight of the solute by the weight of the solution and then converting to a percentage.

II. Methods for the Production of Chitin Nanofibers.

Disclosed herein are methods for the production of chitin nanofibers. In general, the methods disclosed herein for the production of chitin nanofibers comprise dissolving a starting chitin material in a solvent and allowing the self-assembly or formation of the chitin nanofibers. In certain embodiments, the methods disclosed herein for the production of chitin nanofibers comprise dissolving a starting chitin material in a solvent, applying the chitin/solvent solution to a substrate, and allowing the self-assembly of the chitin nanofibers on the substrate. The chitin/solvent solution described herein may also be called a nanofiber ink that is used during the production of chitin nanofibers.

The starting chitin material may be derived from a variety of natural sources. For example, the raw chitin may be collected from cephalopods, crustaceans, mollusks, insects, algae, and fungi and converted into a powdered chitin. The starting chitin material disclosed herein may be provided from commercial sources and may be at least one of an α-chitin, a β-chitin, and a γ-chitin starting material.

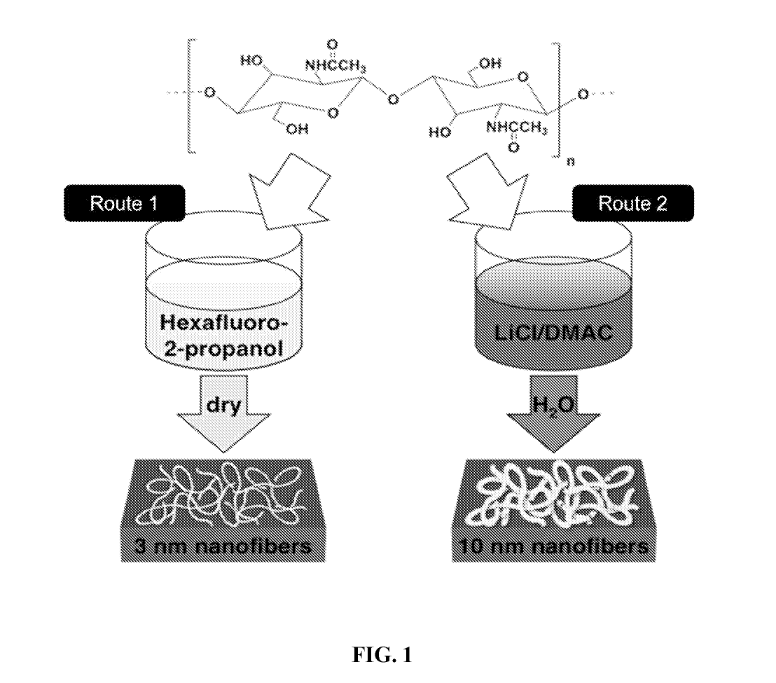

In particular embodiments, the methods disclosed herein for the production of chitin nanofibers comprise dissolving a starting chitin material in an organic solvent. In certain embodiments, as shown by Route 1 in FIG. 1, the organic solvent may include hexafluoro 2-propanol (HFIP). In certain such embodiments, the disclosed methods may comprise dissolving the starting chitin material in an appropriate amount of HFIP to create a chitin/HFIP solution (i.e., chitin/HFIP nanofiber ink) and allowing the formation of chitin nanofibers. In further embodiments, the disclosed methods may comprise dissolving the starting chitin material in an appropriate amount of HFIP to create a chitin/HFIP solution, and placing the chitin/HFIP solution on a substrate, and allowing chitin nanofiber formation by HFIP solvent drying and/or evaporation.

In certain embodiments of the methods for producing chitin nanofibers, the chitin/HFIP solution may be prepared in different concentrations ranging from approximately 0.001 weight percent (wt %) to approximately 10 wt %. In some embodiments, the chitin/HFIP solution may have a concentration of approximately 0.001 wt %, 0.002 wt %, 0.003 wt %, 0.004 wt %, 0.005 wt %, 0.006 wt %, 0.007 wt %, 0.008 wt %, 0.009 wt %, 0.01 wt %, 0.02 wt %, 0.03 wt %, 0.04 wt %, 0.05 wt %, 0.06 wt %, 0.07 wt %, 0.08 wt %, 0.09 wt %, 0.1 wt %, 0.125 wt %, 0.15 wt %, 0.2 wt %, 0.25 wt %, 0.3 wt %, 0.35 wt %, 0.4 wt %, 0.45 wt %, 0.5 wt %, 0.75 wt %, 1.0 wt %, 1.5 wt %, 2.0 wt %, 2.5 wt %, 3.0 wt %, 3.5 wt %, 4.0 wt %, 4.5 wt %, 5.0 wt %, 5.5 wt %, 6.0 wt %, 6.5 wt %, 7.0 wt %, 7.5 wt %, 8.0 wt %, 8.5 wt %, 9.0 wt %, 9.5 wt %, and 10.0 wt %. In other embodiments, the concentration of the chitin/HFIP solution may be used to control the density, dimensions, and morphology of the produced chitin nanofibers. In one such embodiment, concentrations of >0.002 wt % of the chitin/HFIP solution may produce relatively long chitin nanofibers. In another such embodiment, concentrations of >0.002 wt % of the chitin/HFIP solution may produce denser chitin nanofiber structures. In one embodiment, a chitin/HFIP solution with a concentration of ≧0.125 wt % may produce film-like structures comprising randomly aggregated nanofiber networks.

In certain embodiments of the methods disclosed herein for producing chitin nanofibers, the solvent evaporation rates of a chitin/HFIP solution may be adjusted to control the morphology of the produced chitin nanofibers. In such embodiments, the chitin/HFIP solution, or nanofiber ink, may be applied to a substrate before drying and/or evaporation using any appropriate method including drop casting, airbrushing, printing, stamping, painting, writing, etc. The evaporation of the HFIP solvent from the chitin/HFIP solution may occur under ambient conditions or may be controlled by adjusting the temperature, pressure, and humidity during solvent evaporation. In some embodiments, the evaporation time can vary from seconds to days. In particular embodiments, the solvent evaporation rates may be controlled by drying the chitin/HFIP solution with a stream of gas such as air or nitrogen gas (N₂). In other embodiments, the solvent evaporation rates may be controlled by adjusting the temperature, the gas pressure, the type of gas, and changing the solvent partial pressure of the gas.

In particular embodiments of the methods for producing chitin nanofibers, the use of a chitin/HFIP solution may result in individual chitin nanofibers with diameters ranging from approximately 1 nm up to approximately 10 nm. In some embodiments, the use of a chitin/HFIP solution may produce chitin nanofibers with a diameter of approximately 1 nm, 2 nm, 3 nm, 4 nm, 5 nm, 6 nm, 7 nm, 8 nm, 9 nm, and 10 nm. In other embodiments, the use of a chitin/HFIP solution may produce chitin nanofibers with lengths of approximately 1 μm, up to 100 μm, and up to a continuous chitin nanofiber. In certain embodiments, the chitin nanofibers may further assemble in films or components with larger diameters. In further embodiments, the chitin nanofiber produced according to the disclosed methods may comprise individual or composite nanofibers that are an assembly of smaller nanofibers attached to each other.

In other embodiments of the methods of producing nanofibers disclosed herein, the starting chitin material may be dissolved in an organic solvent comprising lithium chloride/N,N-dimethylacetamide (LiCl/DMAC). In such embodiments, as shown by Route 2 in FIG. 1, the disclosed methods may comprise dissolving the starting chitin material in an appropriate amount of LiCl/DMAC to create a chitin/(LiCl/DMAC) solution (i.e., a chitin/(LiCl/DMAC) nanofiber ink) and allowing formation or precipitation of chitin nanofibers by the addition of excess water (or other polar solvent). In other such embodiments, the disclosed methods may comprise dissolving the starting chitin material in an appropriate amount of LiCl/DMAC to create a chitin/(LiCl/DMAC) solution, placing the chitin/(LiCl/DMAC) solution on a substrate, and allowing formation or precipitation of chitin nanofibers by the addition of excess water (or other polar solvent). In certain such embodiments, the self-assembly and precipitation of chitin nanofibers from a chitin/(LiCl/DMAC) solution may include the addition of a volume of water that is approximately 10-25 times more than the original volume of the chitin/(LiCl/DMAC) solution. Ethanol may also be added to the chitin/(LiCl/DMAC) solution to aid the precipitation of the chitin nanofibers. In some embodiments, the chitin/(LiCl/DMAC), or chitin/(LiCl/DMAC) nanofiber ink, may be applied to a substrate using any appropriate method including drop casting, airbrushing, printing, stamping, painting, writing, etc.

In certain embodiments of the methods for producing chitin nanofibers, the chitin/(LiCl/DMAC) solution may be prepared at a desired concentration, for example, at concentrations ranging from approximately 0.01 wt % to approximately 10 wt %. In some embodiments, the chitin/(LiCl/DMAC) solution may have a concentration of approximately 0.01 wt %, 0.02 wt %, 0.03 wt %, 0.04 wt %, 0.05 wt %, 0.06 wt %, 0.07 wt %, 0.08 wt %, 0.09 wt %, 0.1 wt %, 0.125 wt %, 0.15 wt %, 0.2 wt %, 0.25 wt %, 0.3 wt %, 0.35 wt %, 0.4 wt %, 0.45 wt %, and 0.5 wt %, 0.75 wt %, 1.0 wt %, 1.5 wt %, 2.0 wt %, 2.5 wt %, 3.0 wt %, 3.5 wt %, 4.0 wt %, 4.5 wt %, 5.0 wt %, 5.5 wt %, 6.0 wt %, 6.5 wt %, 7.0 wt %, 7.5 wt %, 8.0 wt %, 8.5 wt %, 9.0 wt %, 9.5 wt %, and 10.0 wt %. In other embodiments, the concentration of the chitin/(LiCl/DMAC) solution may be used to control the density, dimensions, and morphology of the produced chitin nanofibers. In one such embodiment, concentrations of >0.02 wt % of the chitin/(LiCl/DMAC) solution may produce relatively long chitin nanofibers.

In particular embodiments of the methods for producing chitin nanofibers, the use of a chitin/(LiCl/DMAC) solution may produce chitin nanofibers with diameters generally larger, on average, than those chitin nanofibers prepared from a chitin/HFIP solution. In such embodiments, the use of a chitin/(LiCl/DMAC) solution may result in chitin nanofibers with diameters ranging from approximately 1 nm up to approximately 50 nm. In certain such embodiments, the use of a chitin/(LiCl/DMAC) solution may produce chitin nanofibers with a diameter of approximately 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, or 50 nm. In further embodiments, the use of a chitin/(LiCl/DMAC) solution may produce chitin nanofibers with lengths of approximately 1 μm, up to 100 μm, and up to a continuous chitin nanofiber. In other embodiments, the chitin nanofibers may further assemble in films or components with larger diameters. In some embodiments, the chitin nanofiber produced according to the disclosed methods may comprise individual or composite nanofibers that are an assembly of smaller nanofibers attached to each other.

III. Methods for the Use of Chitin Nanofibers.

Also disclosed herein are methods for the use of chitin nanofibers in the fabrication of chitin nanofiber structures. In general, chitin nanofiber structures are fabricated by creating a chitin/solvent solution, or chitin nanofiber ink, and allowing the chitin nanofibers to form into a chitin nanofiber structure. In particular embodiments, chitin nanofiber structures are fabricated by applying a chitin/solvent solution, or chitin nanofiber ink, onto a substrate and allowing the formation or self-assembly of the chitin nanofibers on the substrate as disclosed herein. The methods of fabricating chitin nanofiber structures as disclosed herein may be used to fabricate nanofiber structures comprising, for example, a film, aerogel, gel, sponge, foam, 2-dimensional structure, 3-dimensional structure, non-woven fabric, woven fabric, woven filament, non-woven filament, etc. The chitin nanofiber structures as disclosed herein may have multiple potential applications including but not limited to chitin microneedles for wound care devices and drug delivery, biophotonics, tissue adhesive/sutures, scaffolds for artificial organs, and structures for cell culture. In certain embodiments, a chitin nanofiber structure may be fabricated as a protective coating on, for example, metals, surgical tools, implants, car paint, food industry tools, etc. In further embodiments, at least a portion of a chitin nanofiber structure as disclosed herein may be converted into chitosan by deacetylation of the chitin nanofibers. In such embodiments, chitin nanofiber structures may be fabricated that comprise a layer by layer assembly of chitin/chitosan nanostructures.

In particular embodiments, the chitin nanofibers disclosed herein may comprise chitin that is approximately >70% acetylated (i.e., less than 30% deacetylated). In certain such embodiments, the chitin may be approximately 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100% acetylated. Chitosan may be prepared by deacetylation of the N-acetyl glucosamine residues of the chitin polymer. In the preparation of chitosan, the chitin polymer may be partially or completely deacetylated. In certain embodiments, the chitin nanofiber structures described herein may comprise chitosan which can be described as ≦70% acetylated (i.e., at least 30% deacetylated). In certain such embodiments, the chitosan may be approximately 69%, 68%, 67%, 66%, 65%, 64%, 63%, 62%, 61%, 60%, 55%, 50%, 45%, 40%, 35%, 30%, 25%, 20%, 10%, 5%, and 0% acetylated.

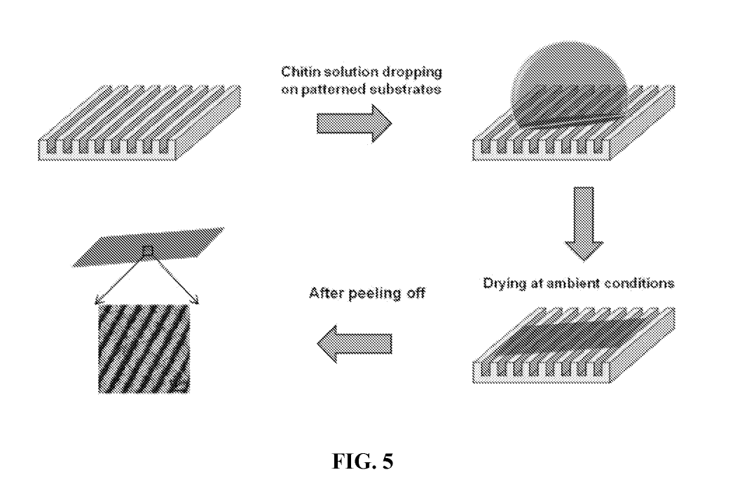

In certain embodiments, chitin nanofiber structures can be fabricated by using the chitin nanofibers disclosed herein in the fabrication of replica molded chitin nanofiber structures. In certain such embodiments, as shown in FIG. 5, replica molded chitin nanofiber structures are fabricated by applying a chitin/solvent solution or chitin nanofiber ink onto a mold or substrate and then allowing the formation or self-assembly of the chitin nanofibers. In particular embodiments, as the chitin nanofiber ink dries, the chitin nanofibers are molded in the shape and/or pattern of at least a portion of the mold or substrate.

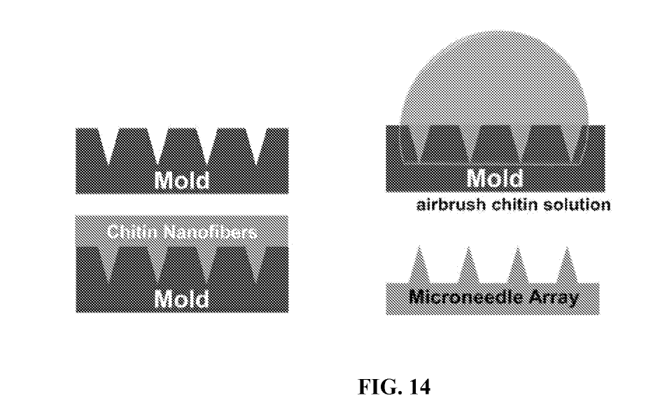

The methods disclosed herein for the fabrication of a replica molded chitin nanofiber structure may include the use of molds or substrates of any material, size, shape, or pattern. In certain embodiments, the mold or substrate may be provided with any desired 2-dimensional shape, 3-dimensional shape, structure or geometry. In particular embodiments, the mold or substrate may be made of metal, plastic, polymer, composite, natural fibers, glass, stone, silicon, silicone, and any other desired material. In further embodiments, the mold or substrate may be grooved, hatched, dimpled, machined, etched, rough, smooth, wavy, etc. In other embodiments, the mold or substrate may be provided with a shape of microneedles, microbrushes, microspheres, nanoneedles, and nanohairs (see, e.g., FIG. 14).

In certain embodiments of the methods of fabricating a replica molded chitin nanofiber structure, the chitin/solvent solution, or chitin nanofiber ink, may be applied to the mold or substrate in a variety of ways. In particular embodiments, the chitin nanofiber ink may be applied to the mold or substrate by drop casting, spraying, airbrushing, pouring, painting, and dipping.

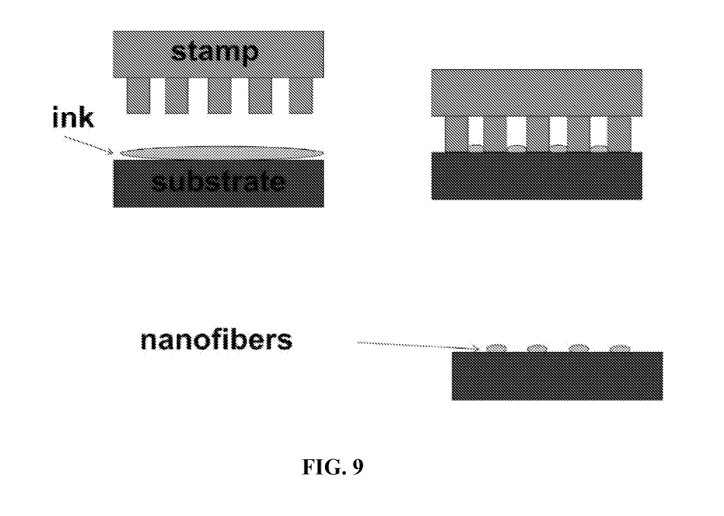

In other embodiments, chitin nanofiber structures can be fabricated by using a micromolding technique. In certain embodiments, as shown in FIG. 9, micromolding comprises placing a chitin/solvent solution or chitin nanofiber ink on a substrate and then a stamp in a desired shape or pattern may be used to mold or pattern the nanofiber ink on the substrate. After drying of the solvent, a chitin nanofiber structure is left on the substrate. The chitin nanofiber structure can then be removed from the substrate or the substrate can be dissolved to leave the chitin nanofiber structure.

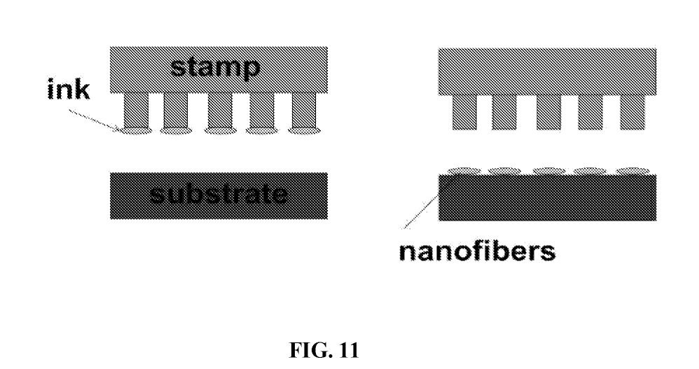

In further embodiments, chitin nanofiber structures can be fabricated by using a printing technique. In such embodiments, as shown by FIG. 11, chitin nanofiber structures are fabricated by using a transfer device such as a patterned device or stamp or similar device to transfer a chitin/solvent solution or chitin nanofiber ink onto a substrate. In certain such embodiments, the stamp may be used to print the nanofiber ink onto the substrate to create a desired shape or pattern in the chitin nanofiber structure. In other embodiments, the chitin nanofiber ink can be printed on any desired substrate using commercially available printers, or any instrument developed for writing such as a pencil, airbrush, pen, marker, dip pen, fountain pen, stamp, ink jet printer, brush, sponge, vaporizer, liquid dispensing device, and aerosol dispensing device, etc. In yet other embodiments, the chitin nanofiber ink may be used in conjunction with 3-dimensional printing devices for direct printing of chitin nanofiber structures.

In certain embodiments of the methods disclosed herein for the fabrication of chitin nanofiber structures, the chitin nanofiber structures may be a chitin nanofiber microneedle array. In such embodiments, the chitin nanofiber microneedle array may be fabricated using any of the techniques disclosed herein including a replica molding technique. In particular embodiments, as shown in FIG. 14, chitin nanofiber microneedle arrays are fabricated by applying a chitin/solvent solution or chitin nanofiber ink onto a microneedle array replica mold and then allowing the formation or self-assembly of the chitin nanofibers. After the chitin nanofiber ink has dried, the chitin nanofiber microneedle array may be removed from the mold and prepared for use.

The embodiments of a chitin nanofiber microneedle array as disclosed herein may be fabricated to be any desired size, dimension, and geometry. In certain embodiments, the chitin nanofiber microneedle array may comprise individual microneedles that have heights of approximately 40 nm up to approximately 3 mm. In particular embodiments, the chitin nanofiber microneedle array may comprise individual microneedles that have heights of approximately 40 nm, 80 nm, 100 nm, 150 nm, 200 nm, 300 nm, 400 nm, 500 nm, 600 nm, 700 nm, 800 nm, 900 nm, 1 μm, 10 μm, 50 μm, 100 μm, 150 μm, 200 μm, 250 μm, 300 μm, 350 μm, 400 μm, 450 μm, 500 μm, 600 μm, 700 μm, 800 μm, 900 μm, 1 mm, 1.5 mm, 2 mm, 2.5 mm, and 3 mm.

In other embodiments, the chitin nanofiber microneedle arrays as disclosed herein may comprise individual microneedles that have widths of approximately 10 nm up to approximately 500 μm. In particular embodiments, the chitin nanofiber microneedle array may comprise individual microneedles that have widths of approximately 10 nm, 20 nm, 30 nm, 40 nm, 50 nm, 60 nm, 70 nm, 80 nm, 90 nm, 100 nm, 125 nm, 150 nm, 175 nm, 200 nm, 300 nm, 400 nm, 500 nm, 600 nm, 700 nm, 800 nm, 900 nm, 1 μm, 10 μm, 25 μm, 50 μm, 75 μm, 100 μm, 125 μm, 150 μm, 175 μm, 200 μm, 225 μm, 250 μm, 300 μm, 350 μm, 400 μm, 450 μm, and 500 μm.

In certain embodiments, the chitin nanofiber microneedle arrays as disclosed herein may comprise individual microneedles that have tips with widths or diameters of approximately 10 nm up to approximately 50 μm. In particular embodiments, the chitin nanofiber microneedle array may comprise individual microneedles that have tips with a width or diameter of approximately 10 nm, 20 nm, 30 nm, 40 nm, 50 nm, 60 nm, 70 nm, 80 nm, 90 nm, 100 nm, 125 nm, 150 nm, 175 nm, 200 nm, 300 nm, 400 nm, 500 nm, 600 nm, 700 nm, 800 nm, 900 nm, 1 μm, 5 μm, 10 μm, 15 μm, 20 μm, 25 μm, 30 μm, 35 μm, 40 μm, 45 μm, and 50 μm.

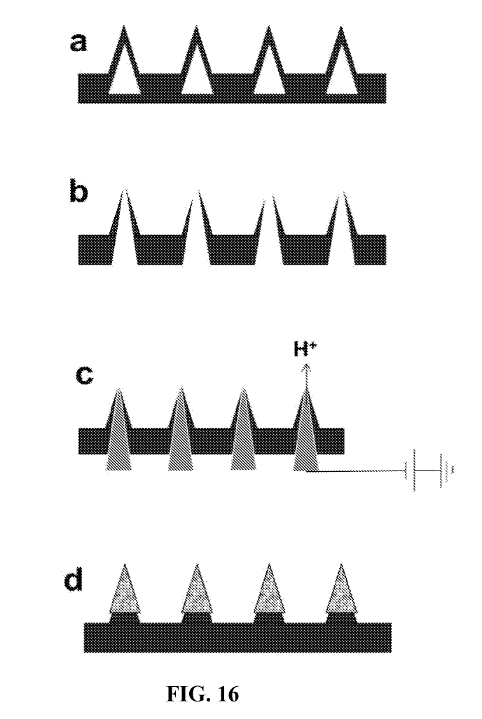

In further embodiments, the chitin nanofiber microneedle arrays as disclosed herein may comprise individual microneedles of a desired shape including the shape of a rod, cone, pyramid, and cylinder. In even further embodiments, the chitin nanofiber microneedle arrays as disclosed herein may comprise individual microneedles that are hollow, barbed, and/or straight. In some further embodiments, the chitin nanofiber microneedle arrays as disclosed herein may comprise individual microneedles that are porous and/or permeable. In still further embodiments, the chitin nanofiber microneedle arrays as disclosed herein may comprise one or more hollow needles with breakable tips and internal reservoirs that may be used to deliver active agents (see, e.g., FIG. 16(a)). In yet further embodiments, the chitin nanofiber microneedle arrays as disclosed herein may comprise one or more internal reservoirs that may be separated by chitosan and may be used as a controlled release or time delayed method of delivery of active agents. In even further embodiments, the chitin nanofiber microneedle arrays as disclosed herein may comprise one or more microneedles that are hollow and may allow delivery of an active agent from an external reservoir, such as a syringe (see, e.g., FIG. 16(b)). In other embodiments, the chitin nanofiber microneedle arrays as disclosed herein may be configured as a biocompatible and/or implantable microelectrode array or H⁺ sensor or H⁺ injector, wherein one or more microneedles include a proton conductor such as a maleic-chitosan or other proton conducting derivative (see, e.g., FIG. 16(c)).

In other embodiments, the chitin nanofiber microneedle arrays as disclosed herein may include one or more individual microneedles with a structure that has been at least partially converted to chitosan. Chitosan may be produced in situ or ex situ by deacetylation of the chitin nanofibers. In still other embodiments, the chitin nanofiber microneedle arrays as disclosed herein may include one or more individual microneedles that comprise a chitosan tip and/or a chitosan base (see, e.g., FIG. 16(d)). In further embodiments, the partial deacetylation of the chitin microneedles may be used to control the mechanical stiffness of the chitin microneedles.

In certain embodiments of a chitin nanofiber microneedle array as disclosed herein, the chitin and/or chitosan may be loaded with or conjugated to a drug, vaccine, imaging agent, or other therapeutic agent, such as an antibiotic, or diagnostic agent, that may be delivered to the tissue of a subject. In particular embodiments of a chitin nanofiber microneedle array as disclosed herein, at least a portion of the chitin nanofiber microneedle array may be loaded with or coated with a cosmetic agent and/or a moisturizing agent. Cosmetic agents include skin-care creams, lotions, powders, perfumes, lipsticks, eye and facial makeup, deodorants, baby products, butters and many other types of products. Moisturizing agents include occlusive agents, humectant agents, emollients, lubricants, silicones, petrolatum, lanolin, wax, propylene glycol, creams, ointments, and other related products.

In some embodiments, when the chitin/chitosan nanofiber microneedle array is applied to the tissue of a subject, the chitin/chitosan nanofibers may dissolve in the tissue and deliver the accompanying drug, vaccine, imaging agent, or other therapeutic or diagnostic agent, to the tissue and/or bloodstream of the subject. In further embodiments, the chitin/chitosan nanofibers may dissolve in the tissue and deliver dissolved chitin and/or chitosan to the tissue and/or bloodstream of the subject.

In some embodiments, the rate that the chitin/chitosan microneedles dissolve may be controlled by adjusting the ratio of chitin and chitosan and/or adjusting the density or shape of the microneedles. In other embodiments, the rate that the chitin/chitosan microneedles dissolve may be controlled, for example, by adjusting the nanofiber diameter, nanofiber surface area, density, porosity, length, degree of entanglement, and the addition of one or more polymers.

In particular embodiments, the chitin nanofiber microneedle arrays may be fabricated with a chitin/HFIP solution, or chitin/HFIP nanofiber ink, as disclosed herein. In such embodiments, the chitin/HFIP solution may be applied to a microneedle array replica mold using any appropriate method including drop casting, airbrushing, printing, stamping, painting, etc. The evaporation of the HFIP solvent from the chitin/HFIP solution may occur under ambient conditions or may be controlled by adjusting the temperature, pressure, and humidity during solvent evaporation. In particular embodiments, the solvent evaporation rates may be controlled by drying the chitin/HFIP solution with a stream of gas such as air or nitrogen gas (N₂).

In other embodiments, the chitin nanofiber microneedle arrays may be fabricated with a chitin/(LiCl/DMAC) solution, or chitin/(LiCl/DMAC) nanofiber ink, as disclosed herein. In such embodiments, the chitin/(LiCl/DMAC) solution may be applied to a microneedle array replica mold using any appropriate method including drop casting, airbrushing, printing, stamping, painting, etc. In certain such embodiments, the chitin/(LiCl/DMAC) solution is applied to the microneedle array replica mold and self-assembly and precipitation of chitin nanofibers is initiated by the addition of excess water.

In further embodiments, the methods for using chitin nanofibers may include the use of patterned chitin nanofiber structures for cell templating and directing of cell growth. In such embodiments, a patterned chitin nanofiber structure may be used as a extracellular matrix analog. In further embodiments, the methods for using chitin nanofibers may include the use of patterned chitin nanofiber structures for neuron cell templating and directing of neuron cell growth. In one such embodiment, a chitin nanofiber tube structure may be used to guide neuron cell growth. The benefits of using patterned chitin nanofiber structures for cell templating may include the biocompatibility of chitin nanofibers, the modeling of the fibril network, the tunable nature of the chitin nanofiber by deacetylation, and use of N-acetylglucosamine as a substrate for glycosaminoglycans (GAGs).

IV. Wound Care Devices Comprising a Chitin Nanofiber Microneedle Array.

Also disclosed herein are wound care devices comprising a chitin nanofiber microneedle array. In certain embodiments of the wound care devices disclosed herein, the wound care devices comprise a bandage or wound dressing including a chitin nanofiber microneedle array for use in wound care. The wound care devices disclosed herein may be flexible, transparent, one-time use products that are biodegradable and amendable to both external and internal use. In some embodiments, the wound care device comprises a chitin nanofiber microneedle array that may be used to close a wound and to promote healing. The wound care devices as disclosed herein may be used to care for all types of wounds including an injury to tissue or skin caused by a cut, surgical procedure, tear, laceration, piercing, trauma or other impact, ulcers, such as diabetic ulcers, ulcers from vascular insufficiency, pressure sores, and burns. In particular embodiments of the wound care devices disclosed herein, the chitin nanofiber microneedles are placed in direct or proximate contact with a wound thereby letting chitin enter the wound and providing the beneficial wound healing effects of chitin. Chitin's beneficial wound healing effects include antibacterial activity, bleeding prevention (hemostasis), decreasing inflammation, reduced adhesions and scarring, water resistance, and breathability. In further embodiments, the wound care devices comprising chitin nanofiber microneedles disclosed herein may be applied to skin and tissue painlessly and provide glueless adhesion and easy removal. In yet further embodiments of the wound care devices disclosed herein, the chitin nanofiber microneedles penetrate the skin and promote the entry of chitin, chitosan, and/or topically applied drugs, such as antibiotics, into the wound.

In some embodiments of wound care devices disclosed herein, the wound care device comprises a chitin nanofiber microneedle array that has been fabricated using a replica mold technique and may be used in conjunction with an additional adhesive. In one such embodiment, a wound care device may comprise a chitin nanofiber microneedle array associated with an adhesive layer that extends at least partially beyond the edges of the chitin nanofiber microneedle array.

In further embodiments, a wound care device as disclosed herein may comprise a chitin nanofiber microneedle array including individual microneedles of a desired shape including the shape of a rod, cone, pyramid, or cylinder. In other embodiments, the wound care devices disclosed herein may include chitin nanofiber microneedle arrays having individual microneedles that are hollow, barbed, or straight. In certain embodiments, the wound care devices disclosed herein may comprise chitin nanofiber microneedle arrays with one or more hollow needles with breakable tips and internal reservoirs that may be used to deliver active agents. In particular embodiments, the wound care devices disclosed herein may comprise chitin nanofiber microneedle arrays having one or more internal reservoirs that may be separated by chitin and/or chitosan and may be used as a controlled release or time delayed method of delivery of active agents. In some embodiments, the wound care devices disclosed herein may comprise chitin nanofiber microneedle arrays with one or more microneedles that are hollow and may allow delivery of an active agent from an external reservoir, such as a syringe. In yet other embodiments, the wound care devices disclosed herein may comprise chitin nanofiber microneedle arrays configured as a biocompatible and/or implantable microelectrode array, H⁺ sensor or H⁺ injector, wherein one or more microneedles include a proton conductor such as a maleic-chitosan or other proton conducting derivative.

In further embodiments, the wound care devices disclosed herein may comprise chitin nanofiber microneedle arrays including one or more individual microneedles that have been at least partially deacetylated and converted to chitosan. In some further embodiments, the wound care devices disclosed herein may comprise chitin nanofiber microneedle arrays with one or more individual microneedles that comprise a chitosan tip and/or a chitosan base. In other further embodiments, a partial deacetylation of the chitin microneedles may be used to control the mechanical stiffness of the chitin microneedles for use in the wound care devices disclosed herein.

In certain embodiments of the wound care devices disclosed herein, the chitin nanofiber microneedles may also comprise chitosan, wherein the chitin and/or chitosan may be loaded with or conjugated to a drug, vaccine, imaging agent, or other therapeutic or diagnostic agent, that may be delivered to the tissue or wound of a subject using a wound care device as disclosed herein. In such embodiments, when the wound care device is applied to the wound of a subject, the chitin/chitosan nanofibers will dissolve in and/or around the wound and deliver the drug, vaccine, imaging agent, chitin, chitosan, or other therapeutic or diagnostic agent, to the wound and/or bloodstream of the subject. In some embodiments of the wound care device disclosed herein, the rate that the chitin/chitosan microneedles dissolve may be controlled by adjusting the ratio of chitin and chitosan and/or adjusting the density or shape of the microneedles. In other embodiments, the rate that the chitin/chitosan microneedles dissolve may be controlled by adjusting the nanofiber diameter, nanofiber surface area, density, porosity, length, degree of entanglement, and the use of additional polymers.

Particular embodiments of the wound care devices disclosed herein may comprise a chitin nanofiber microneedle array that may allow the wound care device to be a glueless bandage that is secured to the skin or tissue. Such embodiments may replace the need for sutures without using glue or other adhesives. In certain such embodiments, the chitin nanofiber microneedle array may comprise one or more microneedles that have a textured surface and/or nano-scale structure that can increase the surface area of the microneedle array and further promote glueless adhesion. In one such embodiment, a glueless wound care device may comprise a chitin nanofiber microneedle array having one or more microneedles approximately 800 nm in height and approximately 200 nm wide.

### EXAMPLES

### Example 1

### Chitin Nanofiber Production from Chitin/HFIP

As represented by Route 1 in FIG. 1, chitin nanofibers were produced according to a solvent evaporation method including a chitin material dissolved in the organic solvent hexafluoro 2-propanol (HFIP) (Sigma Aldrich). Chitin/HFIP solutions ranging from approximately 0.001 to 0.05 wt % chitin/HFIP were prepared by dissolving chitin powder (Industrial Research Ltd., New Zealand) by stirring in HFIP. After dissolving the chitin powder, approximately 5 μL of the chitin/HFIP solution were placed on top of clean silicon wafers and allowed to evaporate at standard temperature and pressure. The average chitin nanofiber yield using this method was >95%.

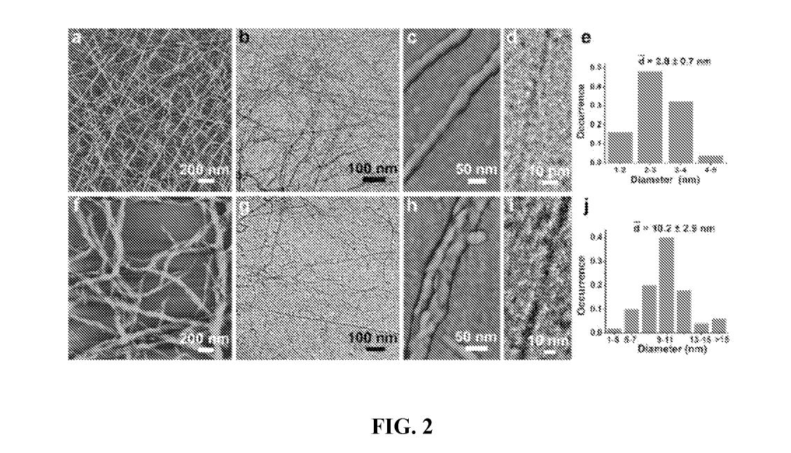

As shown in FIG. 2, micrograph images of the resulting chitin nanofibers showed that chitin nanofibers with an average diameter of 3 nm were produced after solvent evaporation of a 0.01 wt % chitin/HFIP solution. More specifically, FIGS. 2(a)-2(d) shows: (a) atomic force microscopy (AFM) height image, (b) Bright field transmission electron microscopy (TEM) image, (c) AFM phase image of two fibers, (d) TEM image of single nanofiber, (e) nanofiber diameter distribution based on the cross-sectional height profile from AFM height images.

Fourier transformation infrared (FTIR) spectroscopy showed that the resulting chitin nanofibers had chitin chemical structure. Further analysis showed that deacetylation was not observed and that the conditions employed to produce these nanofibers do not cause depolymerization.

Analysis with x-ray diffraction (XRD) showed that the chitin nanofibers are α-chitin with high crystallinity. Further experimentation showed that the self-assembly of chitin in HFIP may be controlled to produce chitin nanofibers with desired characteristics. For example, slowly drying chitin/HFIP solutions of appropriate concentrations leads to relatively long (10-100 μm) and relatively small diameter (2.8±0.7 nm) chitin nanofibers (FIGS. 2(a)-2(d)). Further AFM inspection (FIG. 2(c)) did not detect any surface corrugation suggesting that these materials are composed of a single self-assembled unit.

### Example 2

### Chitin Nanofiber Production from Chitin/(LiCl/DMAC)

As represented by Route 2 in FIG. 1, chitin nanofibers were produced according to a water precipitation method after dissolving chitin material in lithium chloride/N,N-dimethylacetamide (LiCl/DMAC). A 5.0 wt % LiCl/DMAC solution was prepared by dissolving 0.5 grams of LiCl in 10 mL of DMAC. Chitin/(LiCl/DMAC) solutions were prepared by dissolving powdered chitin into the LiCl/DMAC solution. A 0.5 wt % chitin/(LiCl/DMAC) solution was prepared and 20 μL was placed on clean silicon wafers followed by the addition of 500 μL of deionized water. After five seconds, 200 μL of ethanol was added to further the precipitation of the chitin nanofibers onto the silicon wafers. After approximately 1 minute, the silicon wafers were washed with water to remove any remaining LiCl salt. The silicon wafers were then dried under constant N₂ gas flow at standard temperature and pressure. The average yield of chitin nanofibers from this method was 51%.

Microscopic analysis showed that chitin nanofibers with an average diameter of approximately 10 nm were produced from chitin/(LiCl/DMAC) solution using water as a precipitating solvent. More specifically, FIGS. 2(f)-2(j) shows: (f) AFM height image, (g) bright field TEM image, (h) AFM phase image of one nanofiber, (i) TEM image of single nanofiber, and (j) nanofiber diameter distribution based on the cross-sectional height profile from AFM height images. The chitin nanofibers produced according to this method, on the average, have a larger diameter than those chitin nanofibers prepared with HFIP. For the LiCl/DMAC-prepared nanofibers, a complex structure composed of several smaller subunits is discernible in FIGS. 2(f)-2(h). It is possible that chitin nanofiber self-assembly for the two methods proceeds along different pathways.

### Example 3

### Chitin Solution Concentration

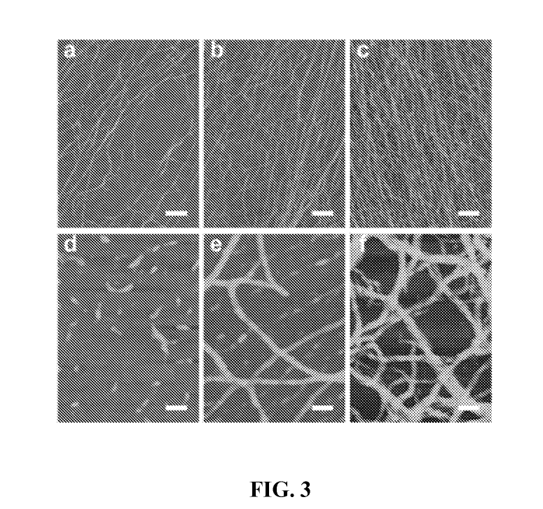

To gather further insight into the nanofiber self-assembly process and nanofiber morphological control, an analysis was made of the chitin nanofibers produced from solutions with different concentrations of chitin (FIG. 3). For each route of chitin nanofiber production, a minimum chitin concentration (>0.002 wt % for HFIP, >0.02 wt % for LiCl/DMAC) was observed to produce long (at least several microns in length) and continuous nanofibers. For the chitin/HFIP route of nanofiber production, chitin solutions above 0.002 wt % concentration had negligible effect on nanofiber dimensions or morphology. As shows in FIGS. 3(a)-3(c) from chitin/HFIP solutions, higher chitin concentrations produced denser nanofiber structures (FIG. 3: (a) 0.005 wt % chitin, (b) 0.01 wt % chitin, and (c) 0.02 wt % chitin). It was estimated from gel formation that approximately 0.5 wt % may be the chitin/solvent concentration upper limit for nanofiber formation from solution.

For the chitin/(LiCl/DMAC) route, higher chitin concentrations produced increasingly dense nanofiber structures (FIG. 3(d) 0.01 wt % chitin, 3(e) 0.02 wt % chitin, and 3(f) 0.2 wt % chitin). However, solutions above 0.02 wt % concentration appeared to have little effect on chitin nanofiber morphology and dimension (not shown).

### Example 4

### Chitin Nanofiber Assembly Kinetics

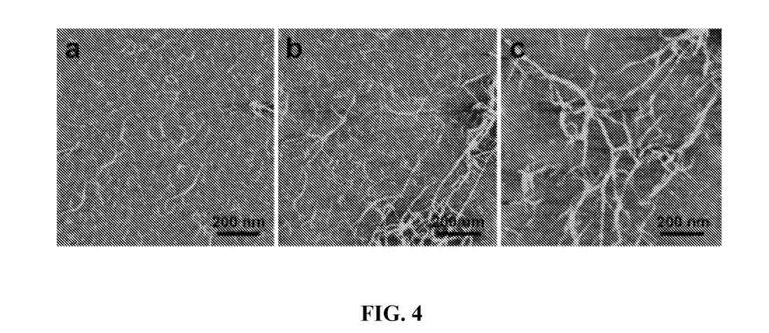

To further study nanofiber assembly kinetics, nanofibers were produced at different solvent evaporation rates. In this example, chitin nanofibers were deposited onto silicon wafers from 5 μL of a 0.002 wt % Chitin/HFIP solutions at different solvent evaporation rates. The results of the different evaporation rates are shown in FIG. 4: (a) the solution was blow dried with N₂ gas (t=3 sec.); (b) the solution was evaporated in ambient air (t=6 sec); (c) the solution was evaporated in a sealed petri dish (t=11 seconds).

For solutions with chitin concentrations >0.002 wt %, evaporation rate did not affect fiber morphology. However, evaporation of a 0.002 wt % solution at twice (3 sec.) the ambient rate (6 sec.) produced short chitin nanofiber stubs that were also observed at lower concentrations. At the same time, doubling the evaporation time (12 sec.) for a 0.002 wt % solution produces nanofibers that are not observed at faster evaporation rates.

These observations suggest that chitin nanofiber assembly may occur at a certain chitin concentration that is reached during the solvent evaporation process. For example, to allow nanofiber formation, a solution of the appropriate chitin concentration may need to exist long enough before the solvent is entirely evaporated. For lower chitin concentrations or rapid evaporation rates, the chitin in the solution may be absorbed onto the substrate before it has a chance to form nanofibers. During a 6 sec. evaporation, a 0.002 wt % chitin/solvent droplet may likely cross a critical volume (ca. 250 times smaller than the original) long enough for the nanofibers to form. The results suggest that chitin nanofiber formation is a relatively fast process that occurs in approximately 1 second and is likely driven by intermolecular hydrogen bonding at a certain chitin concentration. For this example, the solvent HFIP is a strong hydrogen-bond-donor and dissolves chitin through hydrogen bond disruption. However, HFIP is a poor hydrogen-bond-acceptor that does not form any bonds with chitin and is free to quickly evaporate (bp 59° C.) in ambient air.

The results show that self-assembly of chitin nanofibers from a chitin/(LiCl/DMAC) solution follows a different concentration dependence then a chitin/HFIP solution. Specifically, at a concentration of 0.01 wt % chitin/(LiCl/DMAC), short nanofibers approximately 50-100 nm long and approximately 3 nm in diameter were produced (FIG. 3(d)). Similar short nanofiber structures are observed in addition to the long fibrous structures at a concentration of 0.02 wt % (FIG. 2(e)) and disappear when the concentration is greater than 0.2 wt % (FIG. 2(f)).

The production of chitin nanofibers from a chitin/(LiCl/DMAC) solution also showed that chitin fibrous nanostructures that assembled at the concentration of 0.02 wt % comprise large (ca. 10 nm), intermediate (ca. 6 nm), as well as small nanofiber structures (ca. 2.8 nm). Fiber diameter was estimated from fiber height to avoid tip-convolution effects. The average chitin nanofiber diameter is estimated by measuring the height of 50 different individual nanofibers based on the AFM topography image. Intermediate and small nanofibers were observed to adhere to the large nanofiber at several sites. These observations suggest that larger nanofibers observed at higher concentrations are probably formed by self-aggregation of the intermediate and small nanofibers. It is likely that interactions between short nanofibers become more pronounced with increasing chitin solution concentrations and thus eventually drive the spontaneous self-organization of short chitin nanofibers into long chitin nanofibers. It may also be likely that water stabilizes the larger nanofiber structures by, for example, forming intermolecular bridges that promote a higher level of self-organization. This self-aggregation process can be described as nanofiber ripening, which is likely promoted by intermolecular hydrogen bonding and aggregation of the hydrophobic chitin nanostructures in the presence of water.

### Example 5

### Chitin Nanofiber Replica Molding

To demonstrate fabrication of chitin nanofiber structures, various molding templates were used including molding templates made from patterned aluminum sheets or patterned polydimethylsiloxane (PDMS) substrates. Patterned PDMS substrates were prepared by replica molding procedure using the surface of an AFM calibration grating as the master. PDMS preparation was done using SYLGARD 184 elastomer kit; the elastomer base and the curing agent were mixed in 10:1 proportion. After pouring over the master, the PDMS was cured at 100° C. for 1 hour and subsequently the replica was peeled off from the master. Patterned aluminum sheets were directly peeled from blank CD, DVD or Blue-ray disk.

The chitin nanofiber structure fabrication procedure was carried out as depicted in FIG. 5 under ambient conditions by directly dropping 2 mL of either 0.25 wt % or 0.5 wt % chitin/HFIP solution onto the molding templates. The thickness of the chitin nanofiber films can be controlled by adjusting the amount of chitin/HFIP solution used. Upon evaporation of the HFIP solvent, a thin flat film or structure with self-assembled chitin nanofibers was visible on the patterned substrate. Chitin nanofiber based structures of different morphologies were produced using this replica molding technique. The patterned chitin nanofiber structures were peeled off of the molding templates and analyzed.

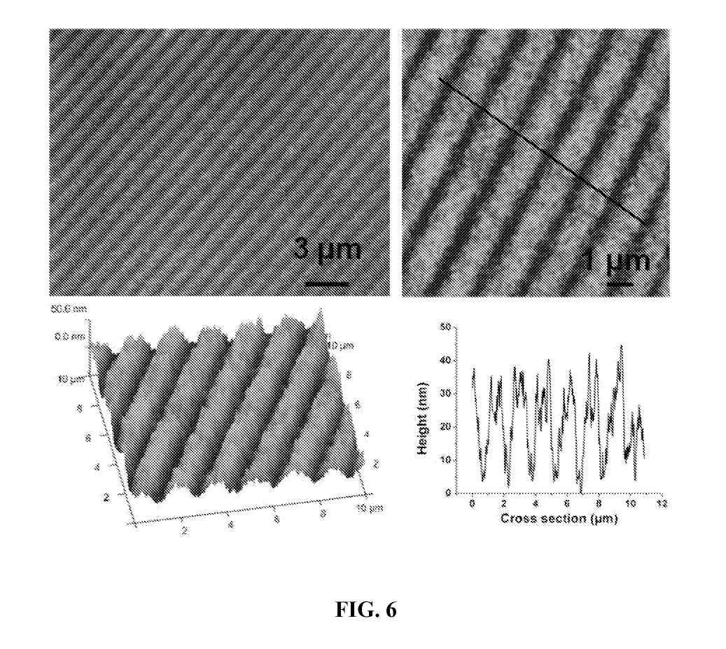

FIG. 6 shows SEM and AFM images of patterned chitin nanofiber films fabricated by replica molding using a 0.5 wt % chitin/HFIP solution that was placed on patterned aluminum sheets peeled from a blank CD disk. The average height of the patterned chitin nanofiber structures was approximately 35 nm. Tapping mode (TM) AFM was performed on a Veeco Multimode V with a Nanoscope IV controller using Veecoprobes Sb-doped Si cantilevers (ρ=0.01-0.025 Ω-cm, k=40 N/m, v˜300 kHz.

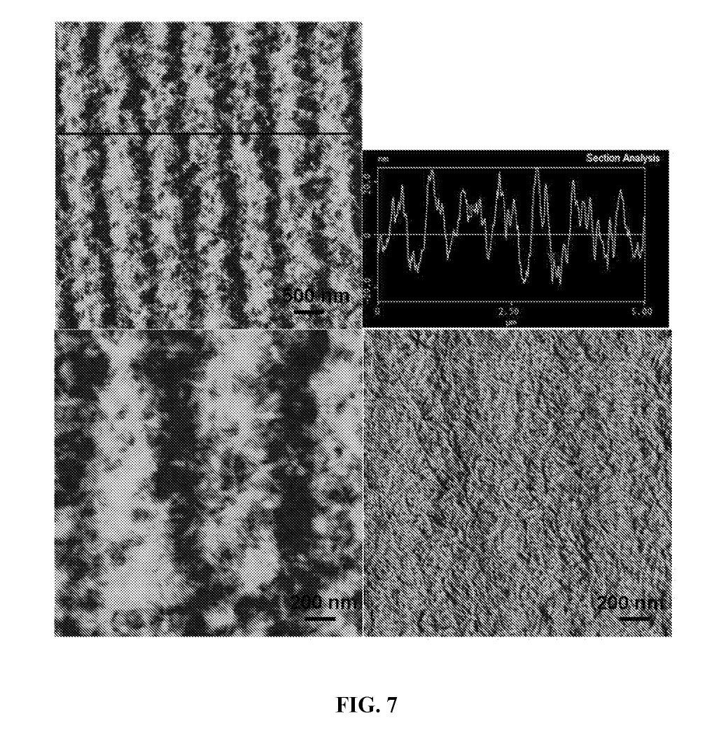

FIG. 7 shows AFM images of patterned chitin nanofiber films fabricated by replica molding using a 0.25 wt % chitin/HFIP solution placed on a patterned aluminum sheet peeled from a blank DVD disk. The average height of the patterned chitin nanofiber structures was approximately 30 nm. The nanofiber feature could be clearly detected in AFM phase image of higher resolution.

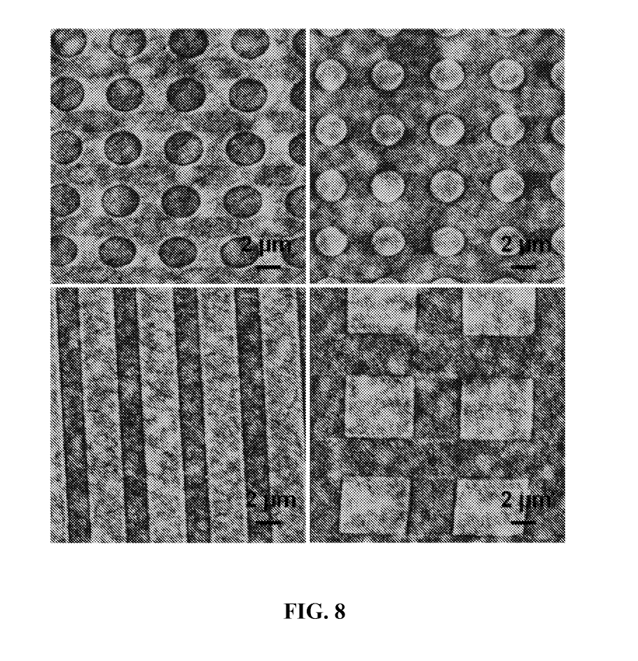

FIG. 8 shows AFM images of patterned chitin nanofiber structures fabricated by replica molding using a 0.25 wt % chitin/HFIP solution placed on patterned PDMS substrates.

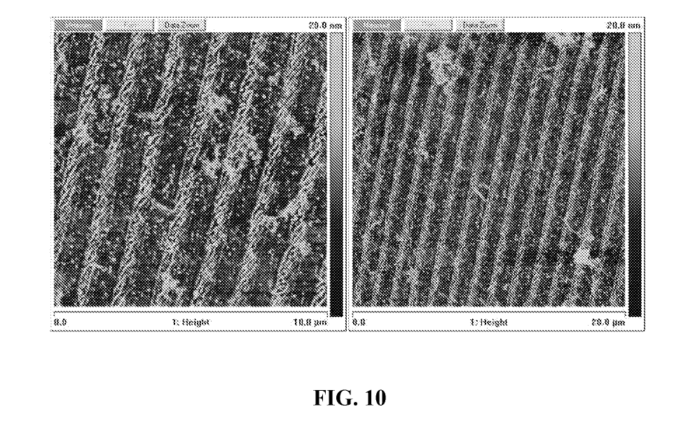

Another chitin nanofiber molding technique is micromolding. For this technique, chitin nanofiber structures or patterns may be formed on a substrate. As shown in FIG. 9, a chitin/HFIP solution or “chitin nanofiber ink” may be placed on a substrate and then a stamp may be used to mold, pattern or form the chitin nanofiber ink on the substrate. After evaporation of the HFIP solvent, a chitin nanofiber structure or pattern is left on the substrate. FIG. 10 shows AFM images of micromolded chitin nanofiber structures in patterned rows that were fabricated using a 0.25 wt % chitin/HFIP solution placed on a glass slide substrate and stamped with a row-patterned stamp.

### Example 6

### Chitin Nanofiber Printing

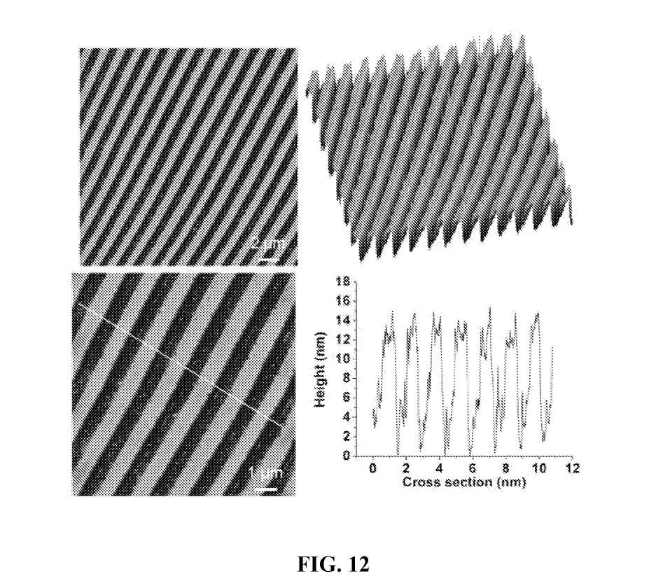

FIG. 11 demonstrates a method of printing chitin nanofiber structures onto a substrate using a patterned stamp to transfer a chitin/solvent solution, or chitin nanofiber ink, onto a substrate. The chitin nanofibers are formed on the substrate in the desired structure or pattern. To further explore this method, chitin nanofibers were printed using row-patterned stamp to print a 0.05 wt % chitin/HFIP solution onto a glass slide substrate. After aging the chitin/HFIP solution on the glass slide for 20 hours, the printed chitin nanofiber structures were visible as well defined rows of chitin nanofibers on the AFM images shown in FIG. 12. The average height of the printed chitin nanofiber structures was approximately 12 nm, and the average width of the rows was approximately 2 nm.

### Example 7

### Chitin Nanofiber Microneedles

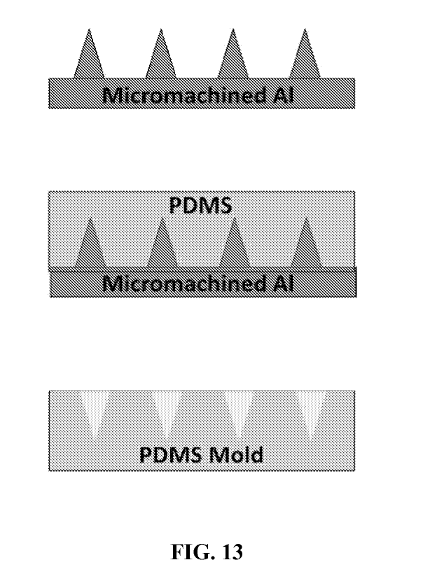

To demonstrate fabrication of chitin nanofiber microneedles, molding templates were formed from micromachined aluminum that were patterned to create a template for a PDMS replica mold for the production of chitin nanofiber microneedle arrays. As diagramed by FIG. 13, micromachined aluminum templates were prepared by machining an aluminum block to fabricate a microneedle array template with each microneedle spike measuring approximately 500 μm high and approximately 250 μm wide at the base of a microneedle spike and approximately 10-50 μm wide at the tip of a microneedle spike. The PDMS mold was fabricated using PDMS replica molding of the silicon microneedle array template. For pouring the PDMS molds, SYLGARD 184 is used with a 10:1 polymer base curing agent ratio. After pouring the liquid PDMS over the micromachined aluminum template, the PDMS was cured at 50° C. for 8 h and then the PDMS mold was separated from the machined aluminum template leaving a PDMS mold for a chitin nanofiber microneedle array.

The chitin nanofiber microneedle array fabrication procedure was carried out as depicted in FIG. 14 under ambient conditions (room temperature in air) by directly pouring the chitin nanofiber ink 0.5% onto the PDMS microneedle array molds and allowing the solution to slowly dry inside a covered container. The chitin nanofiber ink could also be delivered to the microneedle array molds by airbrushing and coat large areas with high throughput. For airbrushing, a commercially available external mix airbrush was used (Paasche H0610) to spray the chitin nanofiber ink onto the microneedle array molds.

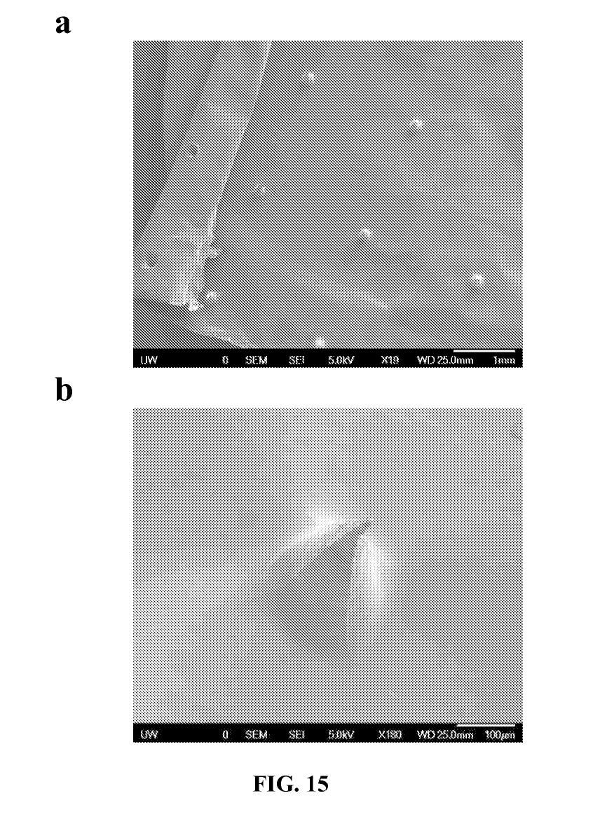

After allowing the formation of the chitin nanofibers and evaporation of the HFIP solvent from the PDMS molds, a chitin nanofiber microneedle array was formed. The chitin nanofiber microneedle arrays were peeled off of the molds and analyzed by SEM as shown in FIGS. 15(a)-(b). FIG. 15(a) shows an SEM image of multiple chitin nanofiber microneedles in an array after being removed from the PDMS mold. FIG. 15(b) shows an SEM image of an individual chitin nanofiber microneedle measuring approximately 500 μm high. The chitin nanofiber microneedle arrays fabricated according this process were mechanically sturdy and could be easily manipulated. Furthermore, the resulting chitin nanofiber microneedle arrays were flexible and transparent. By adjusting the dimensions of the micromachined aluminum templates and the PDMS molds, chitin microneedles were made with lengths ranging from approximately 40 nm up to approximately 3 mm. Other shapes, sizes, and structures of chitin nanofibers could be made by further adjusting the molds to have the desired dimensions.

1. A method of producing chitin nanofibers comprising:
dissolving chitin in a solvent to prepare a chitin/solvent solution; and
allowing or initiating the formation of the chitin nanofibers.

2. (canceled)

3. The method of claim 1, wherein the solvent is hexafluoro 2-propanol (HFIP).

4. The method of claim 1, wherein the chitin/solvent solution is prepared at a concentration selected from a range of approximately 0.001 weight percent (wt %) to approximately 10 wt %.

5. (canceled)

6. The method of claim 1, wherein allowing or initiating the formation of the chitin nanofibers comprises at least one of evaporating the solvent from the chitin/solvent solution and washing the chitin/solvent solution with an excess of a polar solvent.

7. The method of claim 1, wherein the diameter of the chitin nanofibers is at least one of approximately 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, and 50 nm.

8. The method of claim 1, further comprising fabricating a chitin nanofiber structure by allowing or initiating the formation of the chitin nanofibers into a chitin nanofiber structure selected from at least one of a film, aerogel, gel, sponge, foam, 2-dimensional structure, 3-dimensional structure, non-woven fabric, woven fabric, woven filament, and non-woven filament.

9. A method of fabricating a chitin nanofiber structure comprising:
dissolving chitin in a solvent to prepare a chitin/solvent solution;
applying the chitin/solvent solution on a substrate; and
allowing or initiating the formation of the chitin nanofiber structure on the substrate.

10. The method of claim 9, wherein the substrate is a patterned substrate.

11. The method of claim 9, wherein the substrate comprises at least one of a 2-dimensional mold and a 3-dimensional mold.

12. The method of claim 9, wherein the chitin nanofiber structure comprises at least one chitin nanofiber microneedle.

13. The method of claim 9, wherein the chitin nanofiber structure is a chitin nanofiber microneedle array comprising at least two chitin nanofiber microneedles.

14. The method of claim 12, wherein the chitin nanofiber microneedle has a height selected from a range of approximately 40 nm to approximately 3 mm.

15. The method of claim 13, wherein the at least two chitin nanofiber microneedles have widths selected from a range of approximately 10 nm to approximately 500 μm, and heights selected from a range of approximately 40 nm to approximately 3 mm.

16. The method of claim 12, wherein at least a portion of the at least one chitin nanofiber microneedle is chitosan.

17. The method of claim 13, wherein at least a portion of the chitin nanofiber microneedle array is chitosan.

18. A method of fabricating a chitin nanofiber structure comprising:
dissolving chitin in a solvent to prepare a chitin/solvent solution;
applying the chitin/solvent solution to a substrate using a transfer device; and
allowing the formation of the chitin nanofibers on the substrate.

19. The method according to claim 18, wherein the transfer device comprises at least one of a stamp, airbrush, ink jet printer, printer, pen, brush, sponge, vaporizer, liquid dispensing device, and aerosol dispensing device.

20. (canceled)

21. (canceled)

22. A wound care device comprising a chitin nanofiber microneedle array.

23. The wound care device of claim 22, wherein the chitin nanofiber microneedle array comprises chitin nanofibers produced according to the method of claim 1.

24. The wound care device of claim 22, wherein the chitin nanofiber microneedle array comprises microneedles that have widths selected from a range of approximately 10 nm to approximately 500 μm.

25. (canceled)

26. (canceled)

27. (canceled)

28. The wound care device of claim 22, wherein the chitin nanofiber microneedle array comprises at least one porous microneedle.

29. The wound care device of claim 22, wherein the chitin nanofiber microneedle array comprises at least one of a drug, vaccine, imaging agent, therapeutic agent, and diagnostic agent.

30. The wound care device of claim 29, wherein the therapeutic agent comprises at least one antibiotic.

31. (canceled)
